(19) United States
(12) Patent Application Publication
(10) Pub. No.: US 2025/0235258 A1
(43) Pub. Date: Jul. 24, 2025

(54) SEAL ASSEMBLY FOR SURGICAL INSTRUMENTS SUCH AS FOR USE IN SURGICAL ROBOTIC SYSTEMS

(71) Applicant: Covidien LP, Mansfield, MA (US)

(72) Inventors: Haralambos P. Apostolopoulos, Westminster, CO (US); Jason G. Weihe, Longmont, CO (US)

(21) Appl. No.: 18/697,850

(22) PCT Filed: Oct. 28, 2022
(86) PCT No.: PCT/US2022/048271

§ 371 Date: Apr. 2, 2024

Related U.S. Application Data
(63) Continuation-in-part of application No. 63/274,612, filed on Nov. 2, 2021.

(51) Int. Cl. A61B 18/14; A61B 17/00; A61B 17/29; A61B 34/30; A61B 34/37

(52) U.S. Cl. CPC A61B 18/1447; A61B 34/37; A61B 2017/00477; A61B 2017/2948; A61B 2034/302

(57) ABSTRACT

A surgical instrument includes a housing; a shaft extending from the housing and including a proximal segment, a distal segment, and an articulating portion interconnecting the proximal and distal segments; an end effector assembly coupled to the distal segment of the shaft; and a plurality of actuation components extending from the housing into the shaft. Each actuation component extends through the shaft to the articulating portion, the distal segment, or the end effector assembly. A seal assembly is disposed within the proximal segment of the shaft and includes a plug, a clip, and a seal member configured to establish a seal about an internal annular surface of the proximal segment of the shaft. The seal member defines a plurality of apertures therethrough each configured to sealingly receive one of the actuation components. The plug and the clip are configured to engage one another and sandwich the seal member therebetween.

### CROSS-REFERENCE TO RELATED APPLICATION

This application is a 371 National Stage Application of International Application No. PCT/US2022/048271, Oct. 28, 2022, which claims the benefit of, and priority to, U.S. Provisional Patent Application No. 63/274,612, filed on Nov. 2, 2021, the entire contents of which are hereby incorporated herein by reference.

### FIELD

The present disclosure relates to surgical instruments and, more specifically, to a seal assembly for surgical instruments such as, for example, for use in surgical robotic systems.

### BACKGROUND

Surgical robotic systems are increasingly utilized in various different surgical procedures. Some surgical robotic systems include a console supporting a robotic arm. One or more different surgical instruments may be configured for use with the surgical robotic system and selectively mountable to the robotic arm. The robotic arm provides one or more inputs to the mounted surgical instrument to enable operation of the mounted surgical instrument.

The surgical instruments or portions thereof may be configured as single-use instruments or portions that are discarded after use, or may be configured as reusable instruments or portions that are cleaned and sterilized between uses. Regardless of the configurations of the surgical instruments, the console and robotic arm are capital equipment configured for long-term, repeated use. The console and robotic arm may be protected by a sterile barrier during use and/or wiped clean after use to ensure cleanliness for subsequent uses.

### SUMMARY

As used herein, the term “distal” refers to the portion that is being described which is further from an operator (whether a human surgeon or a surgical robot), while the term “proximal” refers to the portion that is being described which is closer to the operator. Terms including “generally,” “about,” “substantially,” and the like, as utilized herein, are meant to encompass variations, e.g., manufacturing tolerances, material tolerances, use and environmental tolerances, measurement variations, design variations, and/or other variations, up to and including plus or minus 10 percent.

In order to inhibit tissue, fluid, and/or debris from traveling from a more-distal portion of a surgical instrument to a more-proximal portion thereof, the present disclosure provides a seal assembly and surgical instruments including the same. The seal assembly of the present disclosure helps to prevent contamination of capital equipment and other surgical instruments or portions thereof disposed proximally of the seal assembly. To the extent consistent, any of the aspects described herein may be used in conjunction with any or all of the other aspects described herein.

Provided in accordance with aspects of the present disclosure is a surgical instrument including a housing and a shaft extending distally from the housing. The shaft includes a proximal segment, a distal segment, and an articulating portion interconnecting the proximal and distal segments. An end effector assembly is coupled to the distal segment of the shaft. A plurality of actuation components extend from the housing into the shaft. Each actuation component of the plurality of actuation components extends through the proximal segment of the shaft to the articulating portion of the shaft, the distal segment of the shaft, or the end effector assembly. A seal assembly is disposed within the proximal segment of the shaft. The seal assembly includes a plug, a clip, and a seal member. The seal member is configured to establish a seal about an internal annular surface of the proximal segment of the shaft and defines a plurality of apertures each configured to sealingly receive one actuation component of the plurality of actuation components therethrough. The plug and the clip are configured to engage one another and sandwich the seal member therebetween.

In an aspect of the present disclosure, the clip includes at least one locking finger defining a tab at a free end thereof. The at least one locking finger is configured to extend through a corresponding passageway defined within the seal member and a corresponding passageway defined within the plug such that the tab is capable of engaging an opening defined within the plug to thereby engage the plug and the clip with one another.

In another aspect of the present disclosure, the seal member is configured to establish a seal about the at least one locking finger extending through the corresponding passageway.

In another aspect of the present disclosure, the seal member includes a body defining an annular rim and at least one annular ring that protrudes radially outwardly from the annular rim. The at least one annular ring is configured to facilitate sealing of the seal member about the internal annular surface of the proximal segment of the shaft.

In still another aspect of the present disclosure, the seal member defines a central aperture configured to receive a first actuation component of the plurality of actuation components and upper and lower passageways spaced above and below the central aperture in vertical registration therewith. The upper and lower passageways are configured to receive portions of the clip to enable engagement of the plug and the clip with one another.

In yet another aspect of the present disclosure, the seal member is configured to establish a seal about the first actuation component extending through the central aperture.

In still yet another aspect of the present disclosure, the central aperture defines an enlarged distal portion compared to a proximal portion thereof.

In another aspect of the present disclosure, the seal member defines a central aperture configured to receive a first actuation component of the plurality of actuation components, a pair of second apertures offset on a first side of the central aperture in vertical registration with one another, and a third aperture offset on a second, oppose side of the central aperture in horizontal registration with the central aperture and collectively the pair of second apertures. The third aperture is configured to receive a second actuation component of the plurality of actuation components.

In yet another aspect of the present disclosure, each second aperture of the pair of second apertures is configured to receive an electrical lead wire therethrough. In such aspects, the seal member may be configured to establish a seal about the electrical lead wires extending through the second apertures and/or the second apertures may define varying diameters along lengths thereof.

In another aspect of the present disclosure, the seal member is configured to establish a seal about the first actuation component extending through the central aperture and about the second actuation component extending through the third aperture.

In still another aspect of the present disclosure, four additional apertures extend through the seal member. Each additional aperture of the four additional apertures is disposed in a different quadrant of the seal member. Each additional aperture may be configured to sealingly receive an additional actuation component of the plurality of actuation components. A diameter of each additional aperture may be tapered along a length thereof.

In another aspect of the present disclosure, a support tube is secured to the clip and extends proximally therefrom. One actuation component of the plurality of actuation components extends through the support tube.

In still yet another aspect of the present disclosure, the clip and the seal member include at least one pair of complementary locating features configured to facilitate proper alignment of the seal member and the clip with one another. In such aspects, one actuation component of the plurality of actuation components may extend through one pair of complementary locating features of the at least one pair of complementary locating features.

In another aspect of the present disclosure, the seal member is overmolded onto the clip.

In another aspect of the present disclosure, the articulating portion of the shaft includes at least one articulating link. In such aspects, the plug may be configured to engage a proximal-most articulating link of the at least one articulating link.

### BRIEF DESCRIPTION OF THE DRAWINGS

The above and other aspects and features of this disclosure will become more apparent in view of the following detailed description when taken in conjunction with the accompanying drawings wherein like reference numerals identify similar or identical elements.

FIG. 1 is a schematic illustration of a surgical robotic system including a control tower, a console, and one or more surgical robotic arms according to aspects of this disclosure;

FIG. 2 is a perspective view of a surgical robotic arm of the surgical robotic system of FIG. 1 according to aspects of this disclosure;

FIG. 3 is a perspective view of a setup arm with the surgical robotic arm of the surgical robotic system of FIG. 1 according to aspects of this disclosure;

FIG. 4 is a schematic diagram of a computer architecture of the surgical robotic system of FIG. 1 according to aspects of this disclosure;

FIG. 5 is a perspective view of a surgical instrument provided in accordance with the present disclosure configured for mounting on a robotic arm of a surgical robotic system such as the surgical robotic system of FIG. 1;

FIGS. 6A and 6B are front and rear perspective views, respectively, of a proximal portion of the surgical instrument of FIG. 5, with an outer shell removed;

FIG. 7 is a front perspective view of the proximal portion of the surgical instrument of FIG. 5 with the outer shell and additional internal components removed;

FIG. 8A is a longitudinal, cross-sectional view of a seal assembly provided in accordance with the present disclosure disposed within a distal portion of a proximal segment of the shaft of the surgical instrument of FIG. 5 and coupled to a proximal-most articulation component of an articulating segment of the shaft of the surgical instrument of FIG. 5;

FIG. 8B is a side view of the seal assembly of FIG. 8A;

FIG. 9A is a perspective view of the seal assembly of FIG. 8A;

FIG. 9B is an exploded, perspective view of the seal assembly of FIG. 8A;

FIG. 10 is a perspective view illustrating the seal assembly of FIG. 8A with the transition plug thereof removed and the seal member overmolded onto the seal clip;

FIG. 11 is an exploded, perspective view of the transition plug of the seal assembly of FIG. 8A and the proximal-most articulation component of the articulating segment of the shaft of the surgical instrument of FIG. 5;

FIGS. 12A-12C are distal face, side, and proximal face views, respectively, of the seal member of the seal assembly of FIG. 8A;

FIG. 12D is a distal face view of the seal member of the seal assembly of FIG. 8A including various operably components of the surgical instrument of FIG. 5 sealingly passing therethrough;

FIGS. 13A, 13B, 13C, and 13D are transverse, cross-sectional views taken across section lines “13A-13A,” “13B-13B,” “13C-13C,” and “13D-13D,” respectively, of FIG. 12A;

FIG. 14 is a side view of the seal clip of the seal assembly of FIG. 8A;

FIG. 15A is a distal face view of the seal clip of the seal assembly of FIG. 8A; and

FIG. 15B is a cross-sectional view taken across section line “15B-15B” of FIG. 15A illustrating the seal member coupled to the seal clip.

### DETAILED DESCRIPTION

This disclosure provides a seal assembly for surgical instruments and surgical instruments including the same. As described in detail below, the seal assembly and surgical instruments of this disclosure are configured for use with a surgical robotic system, which may include, for example, a surgical console, a control tower, and one or more movable carts having a surgical robotic arm coupled to a setup arm. The surgical console receives user input through one or more interface devices, which are interpreted by the control tower as movement commands for moving the surgical robotic arm. The surgical robotic arm includes a controller, which is configured to process the movement command and to generate a torque command for activating one or more actuators of the robotic arm, which, in turn, move the robotic arm in response to the movement command. Those skilled in the art will understand that this disclosure, although described in connection with surgical robotic systems, may also be adapted for use with endoscopic surgical instruments and/or open surgical instruments.

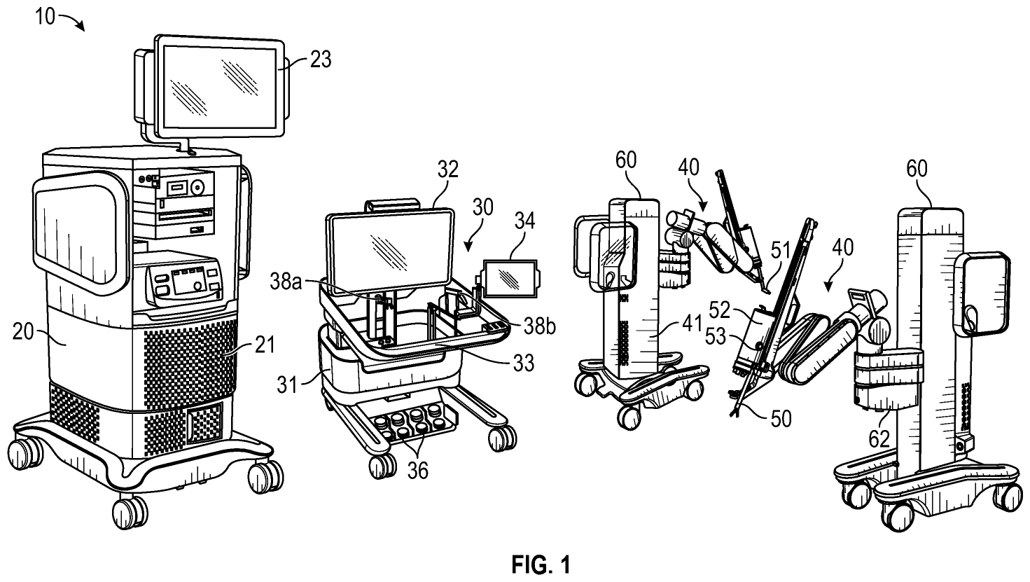

With reference to FIG. 1, a surgical robotic system 10 includes a control tower 20, which is connected to components of the surgical robotic system 10 including a surgical console 30 and one or more robotic arms 40. Each of the robotic arms 40 includes a surgical instrument 50 removably coupled thereto. Each of the robotic arms 40 is also coupled to a movable cart 60.

The one or more surgical instruments 50 may be configured for use during minimally invasive surgical procedures and/or open surgical procedures. In aspects, one of the surgical instruments 50 may be an endoscope, such as an endoscope camera 51, configured to provide a video feed for the clinician. In further aspects, one of the surgical instruments 50 may be an energy-based surgical instrument such as, for example, an electrosurgical forceps or ultrasonic sealing and dissection instrument configured to seal tissue by grasping tissue between opposing structures and applying electrosurgical energy or ultrasonic energy, respectively, thereto. In yet further aspects, one of the surgical instruments 50 may be a surgical stapler including a pair of jaws configured to clamp tissue, deploy a plurality of tissue fasteners, e.g., staples, through the clamped tissue, and/or to cut the stapled tissue.

One of the robotic arms 40 may include a camera 51 configured to capture video of the surgical site. The surgical console 30 includes a first display 32, which displays a video feed of the surgical site provided by camera 51 of the surgical instrument 50 disposed on the robotic arms 40, and a second display 34, which displays a user interface for controlling the surgical robotic system 10. The first and second displays 32 and 34 are touchscreens allowing for displaying various graphical user inputs.

The surgical console 30 also includes a plurality of user interface devices, such as foot pedals 36 and a pair of handle controllers 38a and 38b which are used by a user to remotely control robotic arms 40. The surgical console further includes an armrest 33 used to support clinician's arms while operating the handle controllers 38a and 38b.

The control tower 20 includes a display 23, which may be a touchscreen, and outputs on the graphical user interfaces (GUIs). The control tower 20 also acts as an interface between the surgical console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgical console 30, in such a way that robotic arms 40 and the surgical instrument 50 execute a desired movement sequence in response to input from the foot pedals 36 and the handle controllers 38a and 38b.

Each of the control tower 20, the surgical console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area networks, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DCCP). Wireless communication may be achieved via one or more wireless configurations, e.g., radio frequency, optical, Wi-Fi, Bluetooth® (an open wireless protocol for exchanging data over short distances, using short length radio waves, from fixed and mobile devices, creating personal area networks (PANs)), ZigBee® (a specification for a suite of high level communication protocols using small, low-power digital radios based on the IEEE 122.15.4-2003 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

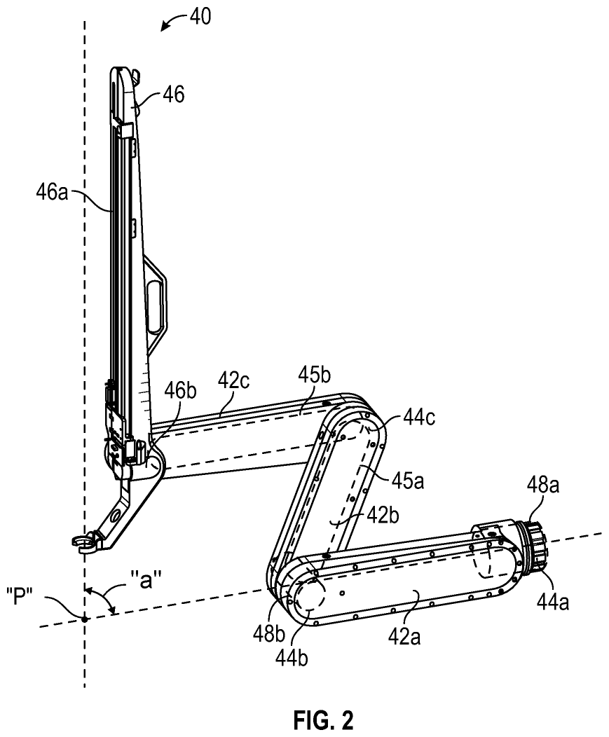

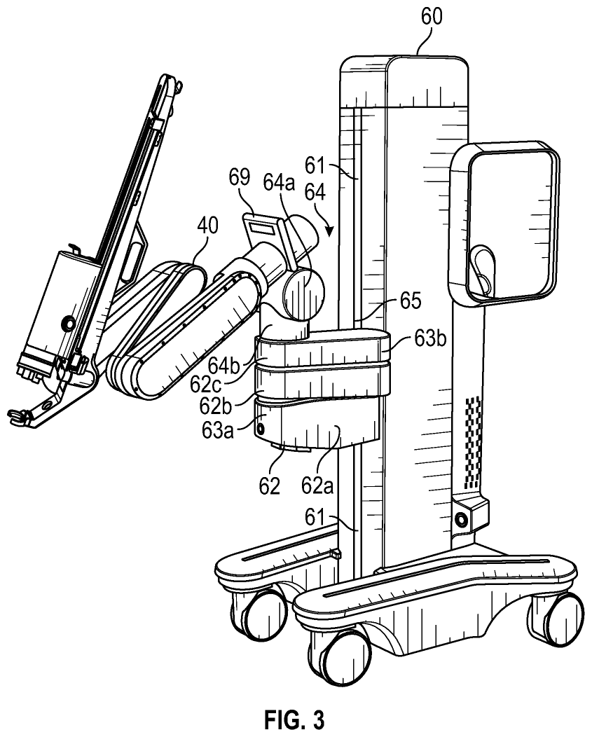

With reference to FIG. 2, each of the robotic arms 40 may include a plurality of links 42a, 42b, 42c, which are interconnected at joints 44a, 44b, 44c, respectively. The joint 44a is configured to secure the robotic arm 40 to the movable cart 60 and defines a first longitudinal axis. With reference to FIG. 3, the movable cart 60 includes a lift 61 and a setup arm 62, which provides a base for mounting of the robotic arm 40. The lift 61 allows for vertical movement of the setup arm 62. The movable cart 60 also includes a display 69 for displaying information pertaining to the robotic arm 40.

The setup arm 62 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 62 includes controls 65 for adjusting movement of the links 62a, 62b, 62c as well as the lift 61.

The third link 62c includes a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

With reference again to FIG. 2, the robotic arm 40 also includes a holder 46 defining a second longitudinal axis and configured to receive an IDU 52 (FIG. 1). The IDU 52 is configured to couple to an actuation mechanism of the surgical instrument 50 and the camera 51 and is configured to move (e.g., rotate) and actuate the instrument 50 and/or the camera 51. IDU 52 transfers actuation forces from its actuators to the surgical instrument 50 to actuate components (e.g., end effectors) of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c.

The robotic arm 40 also includes a plurality of manual override buttons 53 disposed on the IDU 52 and the setup arm 62, which may be used in a manual mode. The clinician may press one or the buttons 53 to move the component associated with the button 53.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

The actuator 48b of the joint 44b is coupled to the joint 44c via the belt 45a, and the joint 44c is in turn coupled to the joint 46c via the belt 45b. Joint 44c may include a transfer case coupling the belts 45a and 45b, such that the actuator 48b is configured to rotate each of the links 42b, 42c and the holder 46 relative to each other. More specifically, links 42b, 42c, and the holder 46 are passively coupled to the actuator 48b which enforces rotation about a remote center point “P” which lies at an intersection of the first axis defined by the link 42a and the second axis defined by the holder 46. Thus, the actuator 48b controls the angle “a” between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle “a.” In embodiments, some or all of the joints 44a, 44b, 44c may include an actuator to obviate the need for mechanical linkages.

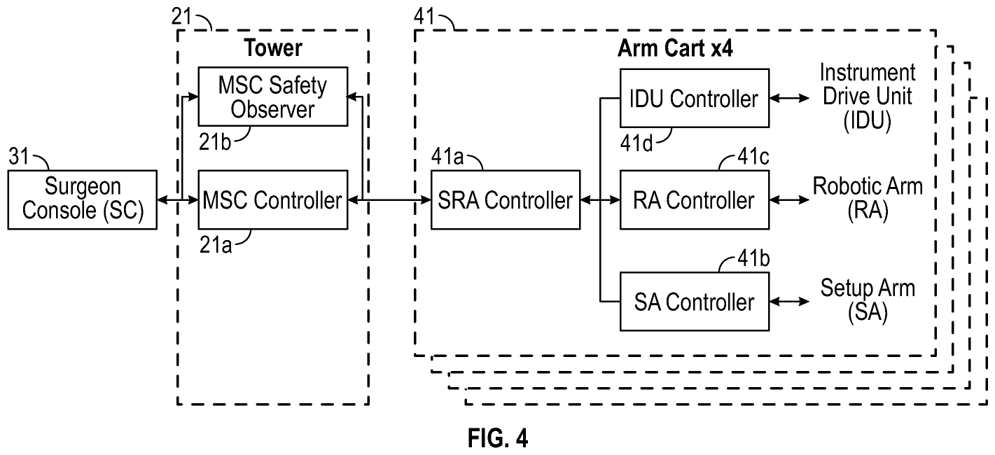

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgical console 30 about the current position and/or orientation of the handle controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives back the actual joint angles and uses this information to determine force feedback commands that are transmitted back to the computer 31 of the surgical console 30 to provide haptic feedback through the handle controllers 38a and 38b. The handle controllers 38a and 38b include one or more haptic feedback vibratory devices that output a haptic feedback. The safety observer 21b performs validity checks on the data going into and out of the controller 21a and notifies a system fault handler if errors in the data transmission are detected to place the computer 21 and/or the surgical robotic system 10 into a safe state.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the movable cart 60, the robotic arm 40, and the IDU 52. The main cart controller 41a also communicates actual joint angles back to the controller 21a.

The setup arm controller 41b controls each of joints 63a and 63b, and the rotatable base 64 of the setup arm 62 and calculates desired motor movement commands (e.g., motor torque) for the pitch axis and controls the brakes. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled as follows. Initially, a pose of the handle controller controlling the robotic arm 40, e.g., the handle controller 38a, is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the handle controller 38a may be embodied as a coordinate position and role-pitch-yaw (“RPY”) orientation relative to a coordinate reference frame, which is fixed to the surgical console 30. The desired pose of the instrument 50 is relative to a fixed frame on the robotic arm 40. The pose of the handle controller 38a is then scaled by a scaling function executed by the controller 21a. In embodiments, the coordinate position is scaled down and the orientation is scaled up by the scaling function. In addition, the controller 21a also executes a clutching function, which disengages the handle controller 38a from the robotic arm 40. In particular, the controller 21a stops transmitting movement commands from the handle controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the handle controller 38a and is then passed by an inverse kinematics function executed by the controller 21a. The inverse kinematics function calculates angles for the joints 44a, 44b, 44c of the robotic arm 40 that achieve the scaled and adjusted pose input by the handle controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

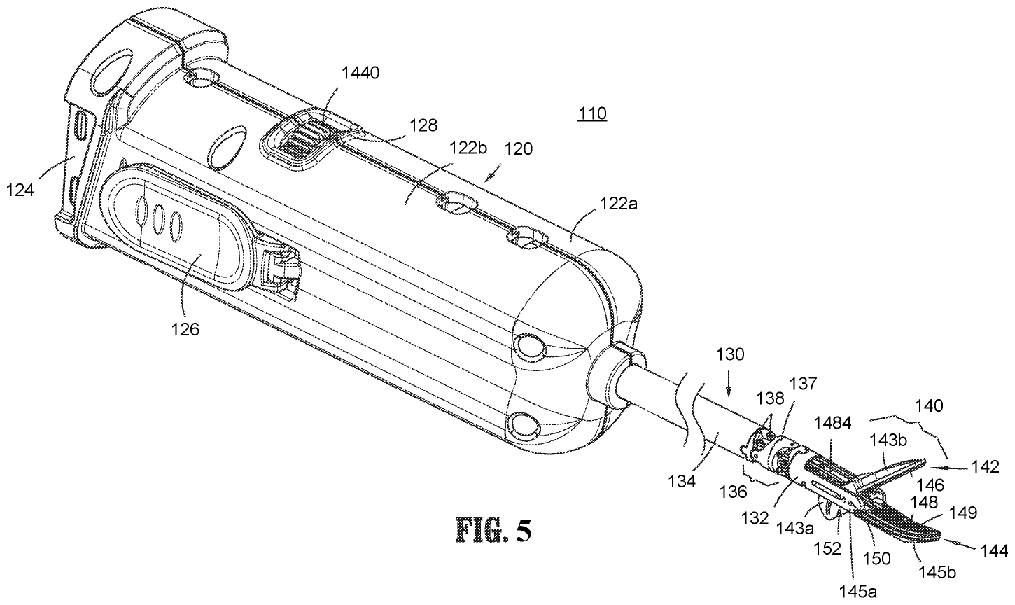

Turning to FIGS. 5-7, a surgical instrument 110 provided in accordance with the present disclosure generally includes a housing 120, a shaft 130 extending distally from housing 120, an end effector assembly 140 extending distally from shaft 130, and an actuation assembly 1100 disposed within housing 120 and operably associated with end effector assembly 140. Instrument 110 is detailed herein as an articulating electrosurgical forceps configured for use with a surgical robotic system, e.g., surgical robotic system 10 (FIG. 1). However, the aspects and features of instrument 110 provided in accordance with the present disclosure, detailed below, are equally applicable for use with other suitable surgical instruments, e.g., graspers, staplers, clip appliers, and/or in other suitable surgical systems, e.g., motorized, other power-driven systems, and/or manually-actuated surgical systems (including handheld instruments).

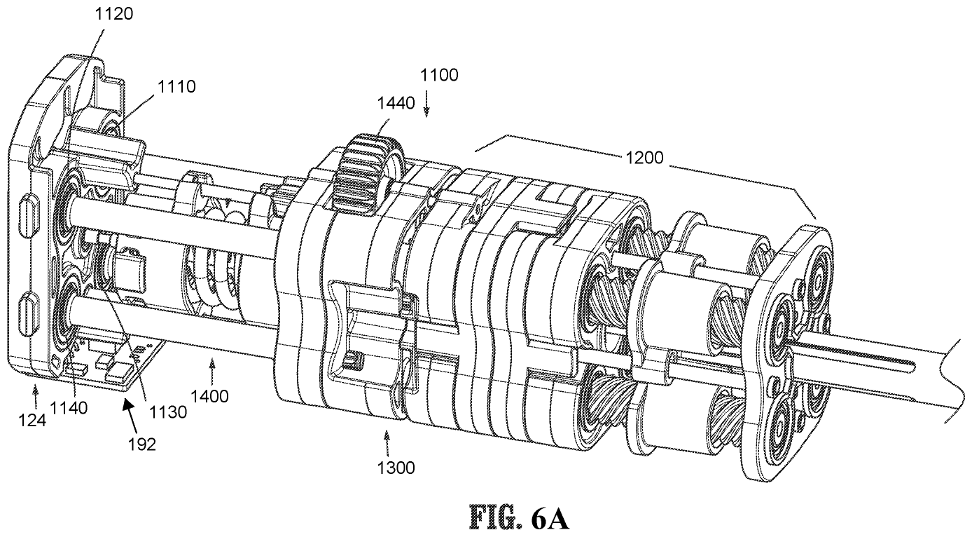

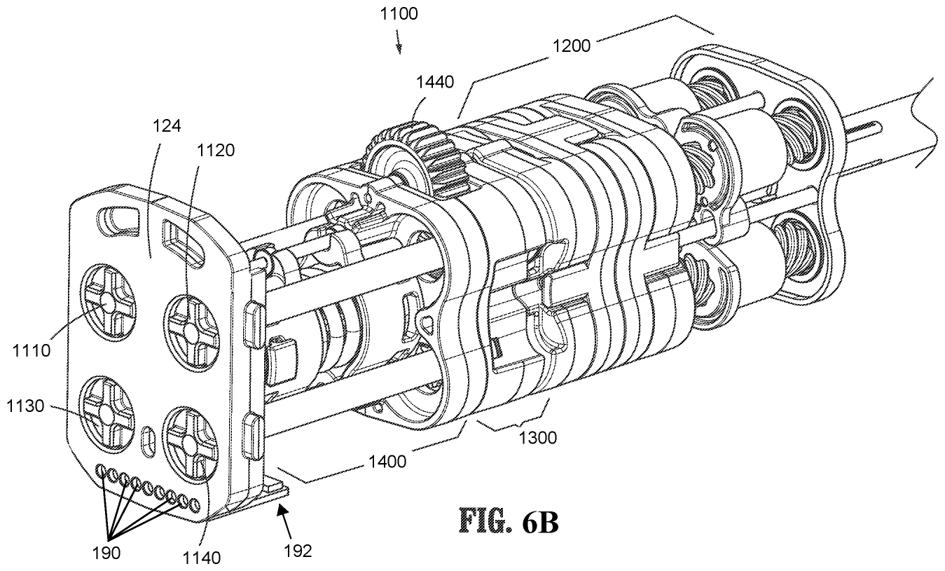

With particular reference to FIG. 5, housing 120 of instrument 110 includes first and second body portion 122a, 122b and a proximal face plate 124 that cooperate to enclose actuation assembly 1100 therein. Proximal face plate 124 includes through-holes defined therein through which input couplers 1110-1140 (FIG. 6B) of actuation assembly 1100 extend. A pair of latch levers 126 (only one of which is illustrated in FIG. 5) extending outwardly from opposing sides of housing 120 enable releasable engagement of housing 120 with a robotic arm of a surgical robotic system, e.g., surgical robotic system 10 (FIG. 1). A window 128 defined through housing 120 permits thumbwheel 1440 to extend therethrough to enable manual manipulation of thumbwheel 1440 from the exterior of housing 120 to permit manual opening and closing of end effector assembly 140.

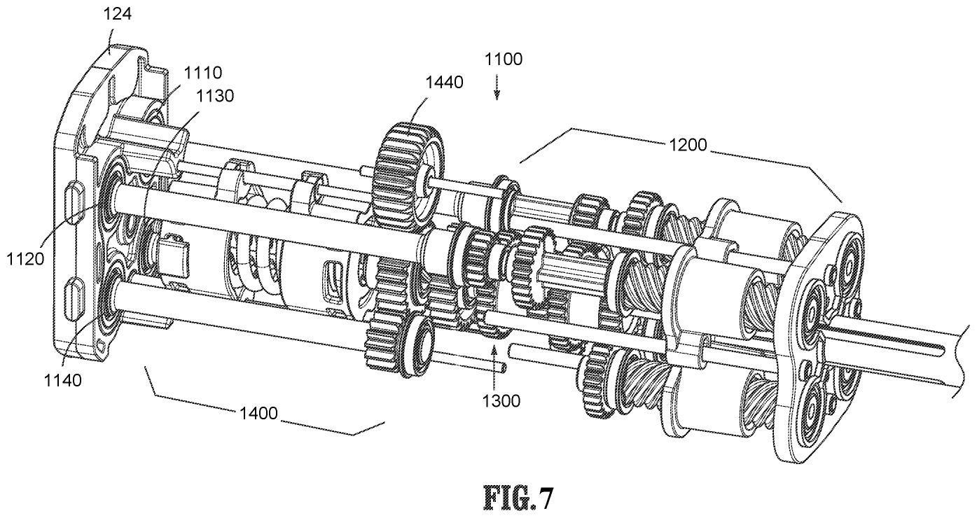

Referring also to FIGS. 6A-7, a plurality of electrical contacts 190 extend through one or more apertures defined through proximal face plate 124 to enable electrical communication between instrument 110 and surgical robotic system 10 (FIG. 1) when instrument 110 is engaged on a robotic arm thereof, e.g., for the communication of data, control, and/or power signals therebetween. As an alternative to electrical contacts 190 extending through proximal face plate 124, other suitable transmitter, receiver, and/or transceiver components to enable the communication of data, control, and/or power signals are also contemplated, e.g., using RFID, Bluetooth®, WiFi®, or via any other suitable wired, wireless, contacted, or contactless communication method. At least some of the electrical contacts 190 are electrically coupled with electronics 192 mounted on an interior side of proximal face plate 124, e.g., within housing 120. Electronics 192 may include, for example, a storage device, a communications device (including suitable input/output components), and a CPU including a memory and a processor. Electronics 192 may be mounted on a circuit board or otherwise configured, e.g., as a chip.

The storage device of electronics 192 stores information relating to surgical instrument such as, for example: the item number, e.g., SKU number; date of manufacture; manufacture location, e.g., location code; serial number; lot number; use information; setting information; adjustment information; calibration information; security information, e.g., encryption key(s), and/or other suitable additional or alternative data. The storage device of electronics 192 may be, for example, a magnetic disk, flash memory, optical disk, or other suitable data storage device.

As an alternative or in addition to storing the above-noted information in the storage device of electronics 192, some or all of such information, e.g., the use information, calibration information, setting information, and/or adjustment information, may be stored in a storage device associated with surgical robotic system 10 (FIG. 1), a remote server, a cloud server, etc., and accessible via instrument 110 and/or surgical robotic system 10 (FIG. 1). In such configurations, the information may, for example, be updated by manufacturer-provided updates, and/or may be applied to individual instruments, units of instruments (e.g., units from the same manufacturing location, manufacturing period, lot number, etc.), or across all instruments. Further still, even where the information is stored locally on each instrument, this information may be updated by manufacturer-provided updates manually or automatically upon connection to the surgical robotic system 10 (FIG. 1).

Referring again to FIG. 5, shaft 130 of instrument 110 includes a distal segment 132, a proximal segment 134, and an articulating section 136 disposed between the distal and proximal segments 132, 134, respectively. Articulating section 136 includes one or more articulating components 137, e.g., links, joints, etc. A plurality of articulation cables 138, e.g., four (4) articulation cables, or other suitable actuators, extend through articulating section 136. More specifically, articulation cables 138 are operably coupled to distal segment 132 of shaft 130 at the distal ends thereof and extend proximally from distal segment 132 of shaft 130, through articulating section 136 of shaft 130 and proximal segment 134 of shaft 130, and into housing 120, wherein articulation cables 138 operably couple with an articulation sub-assembly 1200 of actuation assembly 1100 (FIG. 6A) to enable selective articulation of distal segment 132 (and, thus end effector assembly 140) relative to proximal segment 134 and housing 120, e.g., about at least two axes of articulation (yaw and pitch articulation, for example). Articulation cables 138 are arranged in a generally rectangular configuration, although other suitable configurations are also contemplated. In some configurations, as an alternative, shaft 130 is substantially rigid, malleable, or flexible and not configured for active articulation. Articulation sub-assembly 1200 is described in greater detail below.

With respect to articulation of end effector assembly 140 relative to proximal segment 134 of shaft 130, actuation of articulation cables 138 may be accomplished in pairs. More specifically, in order to pitch end effector assembly 140, the upper pair of cables 138 are actuated in a similar manner while the lower pair of cables 138 are actuated in a similar manner relative to one another but an opposite manner relative to the upper pair of cables 138. With respect to yaw articulation, the right pair of cables 138 are actuated in a similar manner while the left pair of cables 138 are actuated in a similar manner relative to one another but an opposite manner relative to the right pair of cables 138. Other configurations of articulation cables 138 or other articulation actuators are also contemplated.

Continuing with reference to FIG. 5, end effector assembly 140 includes first and second jaw members 142, 144, respectively. Each jaw member 142, 144 includes a proximal flange portion 143a, 145a and a distal body portion 143b, 145b, respectively. Distal body portions 143b, 145b define opposed tissue-contacting surfaces 146, 148, respectively. Proximal flange portions 143a, 145a are pivotably coupled to one another about a pivot 150 and are operably coupled to one another via a cam-slot assembly 152 including a cam pin slidably received within cam slots defined within the proximal flange portion 143a, 145a of at least one of the jaw members 142, 144, respectively, to enable pivoting of jaw member 142 relative to jaw member 144 and distal segment 132 of shaft 130 between a spaced-apart position (e.g., an open position of end effector assembly 140) and an approximated position (e.g., a closed position of end effector assembly 140) for grasping tissue between tissue-contacting surfaces 146, 148. As an alternative to this unilateral configuration, a bilateral configuration may be provided whereby both jaw members 142, 144 are pivotable relative to one another and distal segment 132 of shaft 130. Other suitable jaw actuation mechanisms are also contemplated.

In configurations, a longitudinally-extending knife channel 149 (only knife channel 149 of jaw member 144 is illustrated; the knife channel of jaw member 142 is similarly configured) is defined through the tissue-contacting surface 146, 148 of one or both jaw members 142, 144. In such aspects, a knife assembly including a knife rod (not shown) extending from housing 120 through shaft 130 to end effector assembly 140 and a knife blade (not shown) disposed within end effector assembly 140 between jaw members 142, 144 is provided. The knife blade is selectively translatable through the knife channel(s) 149 and between the jaw member 142, 144 to cut tissue grasped between tissue-contacting surfaces 146, 148 of jaw members 142, 144, respectively. The knife rod is operably coupled to a knife drive sub-assembly 1300 (FIG. 7) of actuation assembly 1100 (FIGS. 6A-6B) at a proximal end thereof to enable the selective actuation of the knife rod to, in turn, reciprocate the knife blade (not shown) between jaw members 142, 144 to cut tissue grasped between tissue-contacting surfaces 146, 148. As an alternative to a longitudinally-advanceable mechanical knife, other suitable mechanical cutters are also contemplated, e.g., guillotine-style cutters, as are energy-based cutters, e.g., RF electrical cutters, ultrasonic cutters, etc., in static or dynamic configurations.

Referring still to FIG. 5, a drive rod 1484 is operably coupled to cam-slot assembly 152 of end effector assembly 140, e.g., engaged with the cam pin thereof, such that longitudinal actuation of drive rod 1484 pivots jaw member 142 relative to jaw member 144 between the spaced-apart and approximated positions. More specifically, urging drive rod 1484 proximally pivots jaw member 142 relative to jaw member 144 towards the approximated position while urging drive rod 1484 distally pivots jaw member 142 relative to jaw member 144 towards the spaced-apart position. However, other suitable mechanisms and/or configurations for pivoting jaw member 142 relative to jaw member 144 between the spaced-apart and approximated positions in response to selective actuation of drive rod 1484 are also contemplated. Drive rod 1484 extends proximally from end effector assembly 140 through shaft 130 and into housing 120 wherein drive rod 1484 is operably coupled with a jaw drive sub-assembly 1400 of actuation assembly 1100 (FIGS. 6A-6B) to enable selective actuation of end effector assembly 140 to grasp tissue therebetween and apply a jaw force within an appropriate jaw force range.

Tissue-contacting surfaces 146, 148 of jaw members 142, 144, respectively, are at least partially formed from an electrically conductive material and are energizable to different potentials to enable the conduction of RF electrical energy through tissue grasped therebetween, although tissue-contacting surfaces 146, 148 may alternatively be configured to supply any suitable energy, e.g., thermal, microwave, light, ultrasonic, ultrasound, etc., through tissue grasped therebetween for energy-based tissue treatment. Instrument 110 defines a conductive pathway (not shown) through housing 120 and shaft 130 to end effector assembly 140 that may include lead wires, contacts, and/or electrically-conductive components to enable electrical connection of tissue-contacting surfaces 146, 148 of jaw members 142, 144, respectively, to an energy source (not shown), e.g., an electrosurgical generator, for supplying energy to tissue-contacting surfaces 146, 148 to treat, e.g., seal, tissue grasped between tissue-contacting surfaces 146, 148.

With additional reference to FIGS. 6A-7, as noted above, actuation assembly 1100 is disposed within housing 120 and includes an articulation sub-assembly 1200, a knife drive sub-assembly 1300, and a jaw drive sub-assembly 1400. Articulation sub-assembly 1200 is operably coupled between first and second input couplers 1110, 1120, respectively, of actuation assembly 1100 and articulation cables 138 (FIG. 5) such that, upon receipt of appropriate inputs into first and/or second input couplers 1110, 1120, articulation sub-assembly 1200 manipulates cables 138 (FIG. 5) to articulate end effector assembly 140 in a desired direction, e.g., to pitch and/or yaw end effector assembly 140. Articulation sub-assembly 1200 is described in greater detail below.

Knife drive sub-assembly 1300 is operably coupled between third input coupler 1130 of actuation assembly 1100 and the knife rod such that, upon receipt of appropriate input into third input coupler 1130, knife drive sub-assembly 1300 manipulates the knife rod to reciprocate the knife blade between jaw members 142, 144 to cut tissue grasped between tissue-contacting surfaces 146, 148.

Jaw drive sub-assembly 1400 is operably coupled between fourth input coupler 1140 of actuation assembly 1100 and drive rod 1484 such that, upon receipt of appropriate input into fourth input coupler 1140, jaw drive sub-assembly 1400 pivots jaw members 142, 144 between the spaced-apart and approximated positions to grasp tissue therebetween and apply a jaw force within an appropriate jaw force range.

Actuation assembly 1100 is configured to operably interface with a surgical robotic system, e.g., system 10 (FIG. 1), when instrument 110 is mounted on a robotic arm thereof, to enable robotic operation of actuation assembly 1100 to provide the above-detailed functionality. That is, surgical robotic system 10 (FIG. 1) selectively provides inputs, e.g., rotational inputs to input couplers 1110-1140 of actuation assembly 1100 to articulate end effector assembly 140, grasp tissue between jaw members 142, 144, and/or cut tissue grasped between jaw members 142, 144. However, as noted above, it is also contemplated that actuation assembly 1100 be configured to interface with any other suitable surgical systems, e.g., a manual surgical handle, a powered surgical handle, etc.

Referring generally to FIGS. 5-7, during use of instrument 110, fluids (blood, other bodily fluids, surgical fluids (including non-liquid fluids such as insufflation gas), air, etc., including fluids carrying tissue, surgical debris, smoke, steam, etc.) from the surgical site may enter instrument 110, e.g., via end effector assembly 140, articulating section 136 of shaft 130, and/or at other locations, and travel proximally within and/or about shaft 130 towards or into housing 120. In order to protect capital equipment such as the robotic arm of the surgical robotic system, e.g., surgical robotic system 10 (FIGS. 1-4), to which instrument 110 is mounted and/or for other purposes such as, for example, to facilitate cleaning all or a portion of instrument 110 in preparation for reuse, to prevent interference with internal operable components, etc., the present disclosure provides a seal assembly disposed at or near articulating section 136 of shaft 130 to inhibit the proximal passage of fluids, tissue, and/or debris proximally into and through surgical instrument 110. However, although the seal assembly of the present disclosure is detailed as located at or near articulating section 136 of shaft 130, it is contemplated that the seal assembly, to the extent practicable, may be used at any other suitable location(s). In addition to protecting capital equipment, the seal assembly of the present disclosure helps maintain insufflation pressure by inhibiting the escape of insufflation gas from the surgical site through surgical instrument 110.

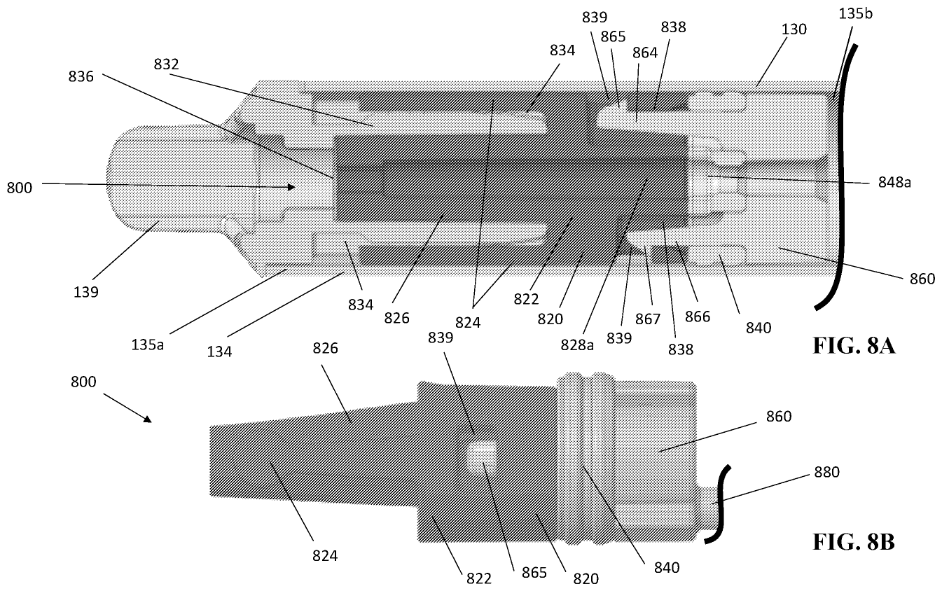

Turning to FIGS. 8A-15B, a seal assembly 800 provided in accordance with the present disclosure is shown disposed within a distal end portion 135a of proximal segment 134 of shaft 130 of surgical instrument 110 (FIG. 5) and operably coupled with a proximal-most articulation link 139 of the one or more articulating components 137 (FIG. 5) of articulating section 136 of shaft 130. Seal assembly 800 is configured to establish a fluid (liquid and/or air-tight) seal within interior volume 135b of proximal segment 134 of shaft 130 and about the internal operable components extending therethrough to inhibit passage of fluids (as well as tissue, debris, etc.) proximally into and through interior volume 135b of proximal segment 134 of shaft 130.

Seal assembly 800 may first be fully assembled and then inserted into distal end portion 135a of proximal segment 134 of shaft 130 (and about the internal operable components therein), the components of seal assembly 800 may be sequentially inserted into distal end portion 135a of proximal segment 134 and into engagement with one another, or seal assembly 800 may be partially assembled and partially sequentially inserted into distal end portion 135a of proximal segment 134. Further, the proximal portion of proximal-most articulation link 139 may be coupled with seal assembly 800 such that the proximal portion of proximal-most articulation link 139 and seal assembly 800 are together inserted into distal end portion 135a of proximal segment 134, or seal assembly 800 may first be installed within distal end portion 135a of proximal segment 134 followed by insertion of the proximal portion of proximal-most articulation link 139 into distal end portion 135a of proximal segment 134 and into engagement with seal assembly 800. Proximal-most articulation link 139, regardless of the order of assembly, may be secured to distal end portion 135a of proximal segment 134 during or after insertion of the proximal portion thereof into distal end portion 135a of proximal segment 134 in any suitable manner such as, for example, via press-fitting, mechanical engagement, adhesion, welding, etc.

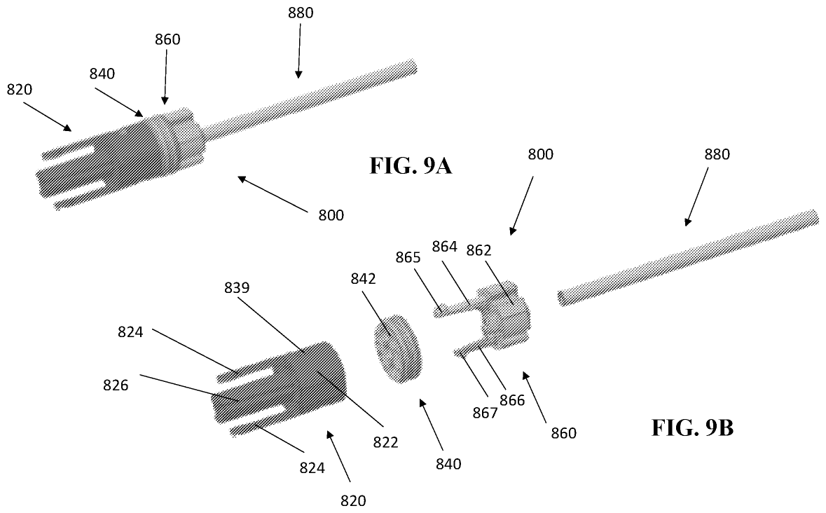

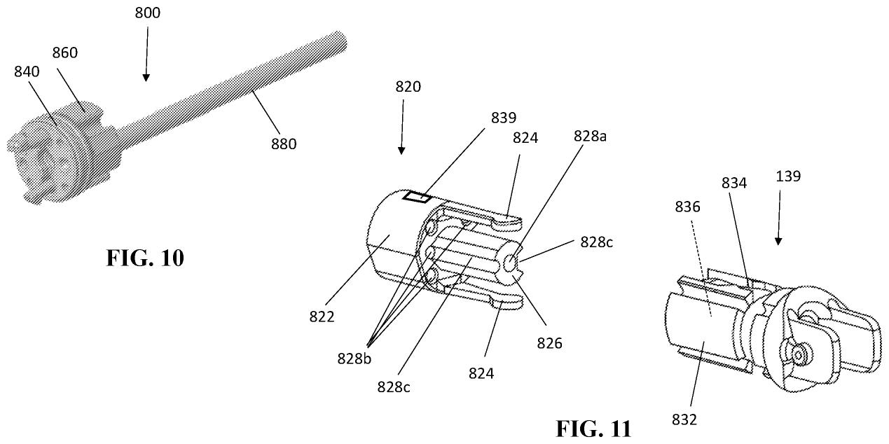

With reference to FIGS. 8A-9B, seal assembly 800 includes a transition plug 820, a seal member 840, a seal clip 860, and a support tube 880. Seal clip 860 is configured to engage transition plug 820, as detailed below, with seal member 840 sandwiched therebetween to thereby retain transition plug 820, seal member 840, and seal clip 860 in engagement with one another inhibiting substantial relative axial or rotational motion therebetween. Seal clip 860 is overmolded or otherwise secured about a distal end portion of support tube 880 in fixed axial and rotational orientation relative thereto. Referring momentarily to FIG. 10, as an alternative to retention of seal member 840 via sandwiching seal member 840 between the engaged seal clip 860 and transition plug 820, seal member 840 may be overmolded onto seal clip 860. In aspects where seal clip 860 is also overmolded about the distal end portion of support tube 880, a two-shot overmold may be utilized wherein seal clip 860 is first overmolded about the distal end portion of support tube 880 (the first shot) and, thereafter, seal member 840 is overmolded onto seal clip 860 (the second shot), although other configurations are also contemplated.

As illustrated in FIGS. 8A-9B and 11, transition plug 820 includes a base 822 configured to proximally abut a proximal body portion 832 of proximal-most articulation link 139 of the one or more articulating components 137 of articulating section 136 of shaft 130 (see FIG. 5), a pair of opposed arms 824 extending distally from base 822, and a central post 826 extending distally from base 822 between arms 824. Arms 824 are configured for engagement within corresponding slots 834 defined within proximal-most articulation link 139 while central post 826 is received within a central cavity 836 defined within proximal-most articulation link 139 to thereby rotationally and axially fix transition plug 820 relative to proximal-most articulation link 139 when transition plug 820 and the proximal portion of proximal-most articulation link 139 are positioned within distal end portion 135a of proximal segment 134 of shaft 130. With transition plug 820 and the proximal portion of proximal-most articulation link 139 positioned within distal end portion 135a of proximal segment 134 of shaft 130, the lack of or minimal clearance within proximal segment 134 inhibits disengagement of arms 824 from slots 834, thereby maintaining the above-detailed engagement between transition plug 820 and proximal-most articulation link 139.

Base 822 and central post 826 of transition plug 820 cooperate to define a central lumen 828a extending therethrough, base 822 includes a plurality of radial lumens 828b extending therethrough and arranged about central lumen 828a, and central post 826 defines a pair of opposed channels 828c on either side of central lumen 828a that cooperate with corresponding radial lumens 828b. The lumens 828a, 828b and channels 828c cooperate to receive the various internal components of surgical instrument 110 (FIG. 5) routed through seal clip 860 and seal member 840 of seal assembly 800. Base 822 of transition plug 820 further includes a pair of radially-opposed proximal passageways 838 (see FIG. 8A) extending therein from the proximal end thereof. Proximal passageways 838 communicate with radial openings 839 disposed through the exterior annular surface of base 822 (see FIGS. 8A and 8B).

Transition plug 820 may be formed from a substantially rigid polymeric material or any other suitable substantially rigid or semi-rigid material. Although transition plug 820 may be formed from a substantially rigid or semi-rigid material, the cantilever configuration of arms 824 enable flexion of arms 824 to ride over proximal-most articulation link 139 and into engagement within slots 834 thereof. In other configurations, transition plug 820 is elastomeric or otherwise formed from a flexible material. In aspects, transition plug 820 does not establish a seal about any components inserted therethrough and/or does not establish a seal within interior volume 135b of proximal segment 134 of shaft 130; rather, seal member 840 provides the necessary seals to inhibit the passage of fluids (including liquids and/or gasses), tissue, and/or debris proximally into and through proximal segment 134 of shaft 130. However, in other aspects, transition plug 820 additionally or alternatively provides some or all of these seals.

Proximal-most articulation link 139 may be formed from a metal, e.g., stainless steel, or any other suitable material, and is configured to pivotably connect to a next articulation component of the one or more articulating components 137 of articulating section 136 of shaft 130 (see FIG. 5) to permit articulation of end effector assembly 140 of surgical instrument 110 (see FIG. 5) relative to proximal segment 134 of shaft 130 in at least one plane. Additional pivotable couplings at different orientations relative to one another may be provided to allow for articulation of end effector assembly 140 of surgical instrument 110 (see FIG. 5) relative to proximal segment 134 of shaft 130 in multiple planes.

Turning to FIGS. 8A-9B and 12A-13D, seal member 840 is formed from an elastomeric material, e.g., via injection molding or in any other suitable manner, and includes a seal body 842 defining a coin-shaped configuration having a proximal face 844a, a distal face 844b, and an annular rim 844c. Seal member 840, more specifically, may be formed from silicone, e.g., an 80 durometer silicone, a 60 durometer silicone, a 55 durometer silicone, or any silicone having a durometer in a range of about 35 to about 95 or, in other aspects, a durometer in a range of about 50 to 95. In aspects, the silicone used to form seal member 840 is self-lubricating, thus eliminating the need to parylene coat seal member 840 to reduce friction. Alternatively or additionally, seal member 840 may be coated with parylene or other suitable lubricant to reduce friction.

Seal body 842 of seal member 840 includes a pair of spaced-apart annular rings 846 that protrude radially outwardly from annular rim 844c to facilitate sealing engagement of seal body 842 of seal member 840 within interior volume 135b of proximal segment 134 of shaft 130, although greater or fewer than two annular rings 846, or other suitable components facilitating sealing, are also contemplated. In aspects, the more-proximal annular ring 846 defines a chamfered proximal edge to facilitate insertion into and sealing within interior volume 135b of proximal segment 134 of shaft 130. Further, seal body 842 defines a plurality of apertures defined therethrough from proximal face 844a to distal face 844b thereof, including: a central jaw drive aperture 848a; upper and lower finger passageways 848b, 848c vertically registered with central jaw drive aperture 848a and radially spaced above and below central jaw drive aperture 848a, respectively; a pair of lead wire apertures 850a, 850b vertically registered with respect to one another and radially spaced from central jaw drive aperture 848a on a first side of central jaw drive aperture 848a; a knife rod aperture 852 radially spaced from central jaw drive aperture 848a on a second, opposite side of central jaw drive aperture 848a in horizontal registration with central jaw drive aperture 848a and collectively the pair of lead wire apertures 850a, 850b; and four articulation cable apertures 854 arranged in a substantially square configuration (although rectangular and other polygonal configurations are also contemplated) wherein each articulation cable aperture 854 is disposed in one of the quadrants of seal body 842 (e.g., a first quadrant above and to the second side of central jaw drive aperture 848a, a second quadrant below and to the second side of central jaw drive aperture 848a, a third quadrant below and to the first side of central jaw drive aperture 848a, and a fourth quadrant above and to the first side of central jaw drive aperture 848a). In aspects, knife rod aperture 852 is initially sealed closed by a thinned portion of material of seal body 842. That is, as manufactured, knife rod aperture 852 is sealed closed and is configured to be punctured to establish knife rod aperture 852 during assembly of surgical instrument 110 (FIG. 5).

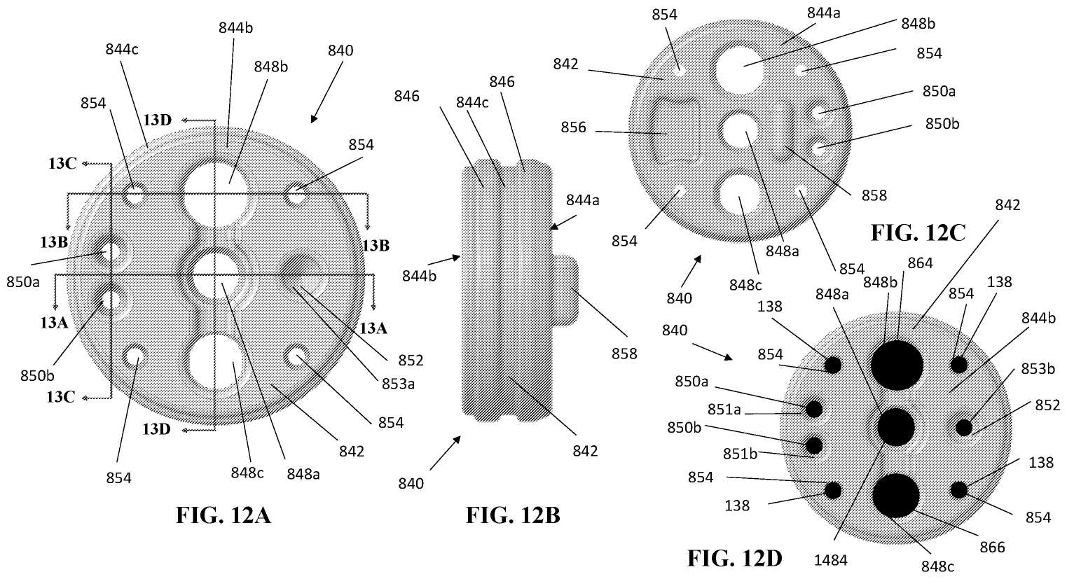

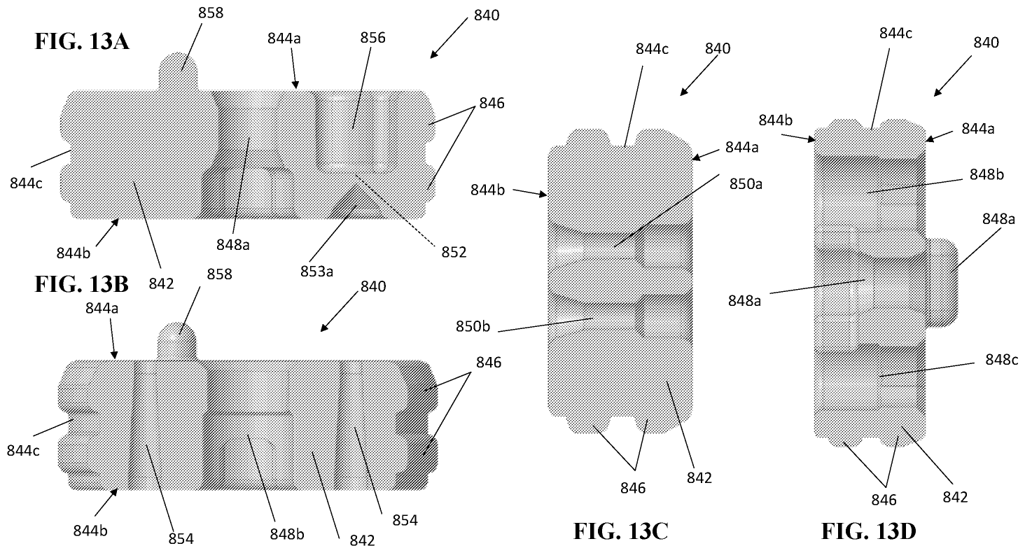

Continuing with reference to FIGS. 12A-13D, seal member 840 further includes one or more locating features 856, 858, e.g., recessed and/or protruding features, defined on or within proximal face 844a thereof and positioned offset from central jaw drive aperture 848a. Locating features 856, 858 may define different shape and/or profile (e.g., recessed vs protruding) configurations to, as detailed below, ensure proper alignment of seal member 840 during assembly of seal assembly 800. In aspects, recessed locating feature 856 includes knife rod aperture 852 defined therethrough. The reduced-thickness of seal body 842 of seal member 840 within recessed locating feature 856 thus provides a thinned portion of material to facilitate puncture to establish knife rod aperture 852. A cut-out 853a may be defined partially through distal face 844b of seal body 842 to, in cooperation with recessed locating feature 856, further thin the portion of material to be punctured to define knife rod aperture 852.

Referring in particular to FIG. 12D, the various apertures of seal member 840 are configured to receive and sealingly engage the operable components of surgical instrument 110 (FIG. 5) that extend through seal assembly 800 (FIGS. 8A-9B) to operably to end effector assembly 140 (FIG. 5), as detailed below. For example, central jaw drive aperture 848a is configured to receive and sealingly engage jaw drive rod 1484 (or other suitable jaw drive component or surrounding component, e.g., a jaw drive tube or supporting tube disposed about jaw drive rod 1484). As illustrated in FIGS. 13A and 13B, in conjunction with FIG. 12D, central jaw drive aperture 848a may define a body, a flared proximal end portion, and an enlarged distal end portion to facilitate insertion of jaw drive rod 1484, sliding of jaw drive rod 1484 with reduced friction, and manipulation of jaw drive rod 1484 without breaking the seal therebetween.

Upper and lower finger passageways 848b, 848c are configured to receive and sealingly engage locking fingers 864, 866 of seal clip 860, respectively. Upper and lower finger passageways 848b, 848c define different diameters as do locking fingers 864, 866 to ensure correct orientation of seal clip 860 and seal member 840 relative to one another upon engagement therebetween. As illustrated in FIG. 13D, in conjunction with FIG. 12D, upper and lower finger passageways 848b, 848c may define relatively narrowed proximal portions and relatively enlarged distal portions to facilitate insertion of locking fingers 864, 866 with reduced friction while maintaining the seal therebetween.

Lead wire apertures 850a, 850b are configured to sealingly engage electrical lead wires 851a, 851b, respectively, that extend through shaft 130 to electrically connect tissue-contacting surfaces 146, 148 of jaw members 142, 144, respectively, to an energy source (not shown) (see FIG. 5). As illustrated in FIG. 13C, in conjunction with FIG. 12D, lead wire apertures 850a, 580b may define bodies, flared proximal end portions, and enlarged distal end portions to facilitate insertion of electrical lead wires 851a, 851b, and sliding and manipulation of electrical lead wires 851a, 851b with reduced friction without breaking the seal therebetween.

Knife rod aperture 852 is configured to receive (and be formed via puncturing) and sealingly engage knife rod 853b. As illustrated in FIG. 13A, in conjunction with FIG. 12D, and as noted above, knife rod aperture 852 is formed by recessed locating feature 856, cut-out 853a, and a puncturable thinned portion of material. Cut-out 853a may define a conical-shaped configuration to facilitate insertion of knife rod 853b into knife rod aperture 852 and puncturing of the thinned portion of material therewith. As knife rod 853b punctures the thinned portion of material, a seal is formed about knife rod 853b. Piercing the thinned portion to form a seal about knife rod 853b reduces friction by reducing tolerances by putting the seal hole on the centerline of knife rod 853b. Further, although described with respect to sealing about knife rod 853b, thinned portions of material of seal member 840 may be pierced by any other components to additionally or alternatively establish the seals thereabout.

Each articulation cable aperture 854 is configured to receive and sealingly engage one of articulation cables 138. As illustrated in FIG. 13B, in conjunction with FIG. 12D, cable apertures 854 may taper in diameter in a distal-to-proximal direction and be configured to seal articulation cables 138 along substantially the entire lengths of apertures 854 while still permitting sliding and manipulation of articulation cables 138 with reduced friction and without breaking the seal therebetween. The taper of cable apertures 854 in the distal-to-proximal direction may result in a reduction in diameters of cable apertures 854 of from about 20% to about 100% across the lengths of apertures 854 in the distal-to-proximal direction; in other aspects, from about 30% to about 80%; in still other aspects, from about 40% to about 70%.

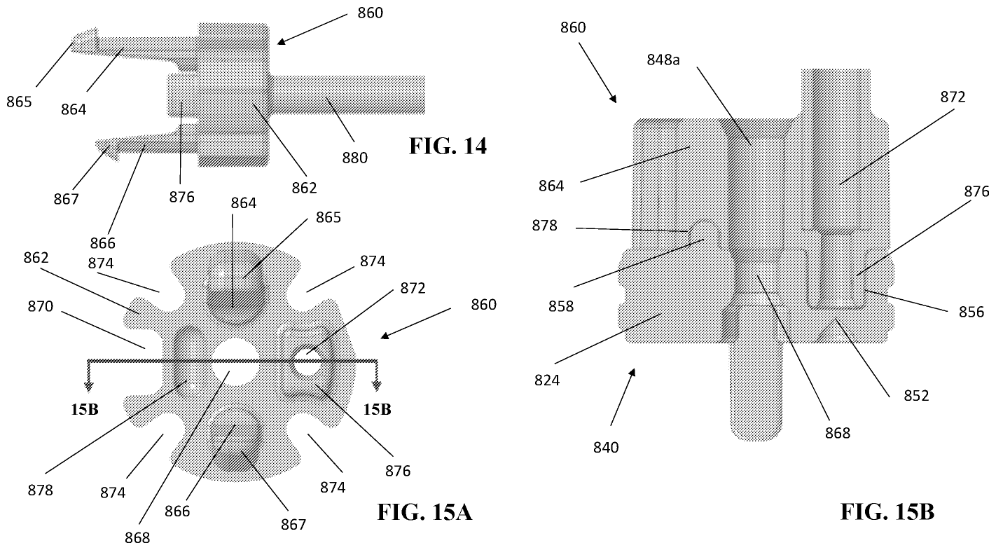

Turning to FIGS. 14-15B, seal clip 860 includes a clip body 862 and relatively large and small locking fingers 864, 866 extending distally from clip body 862 and including locking tabs 865, 857, respectively, disposed at the free ends thereof. Seal clip 860, as mentioned above, also includes support tube 880 secured thereto and extending proximally therefrom.

Seal clip 860 may be formed from a substantially rigid polymeric material or any other suitable substantially rigid or semi-rigid material. Although seal clip 860 may be formed from a substantially rigid or semi-rigid material, the cantilever configuration of fingers 864, 866 enables flexion of fingers 864, 866 to facilitate engagement thereof with transition plug 820, as detailed below. In other configurations, seal clip 860 is elastomeric or otherwise formed from a flexible material. In aspects, seal clip 860 does not establish a seal about any components inserted therethrough and/or does not establish a seal within interior volume 135b of proximal segment 134 of shaft 130; rather, seal member 840 provides the necessary seals to inhibit the passage of fluids (liquid and/or gas), tissue, and/or debris proximally into and through proximal segment 134 of shaft 130, as detailed above. However, in other aspects, seal clip 860 additionally or alternatively provides some or all of these seals.

With additional reference to FIGS. 8A and 8B, locking fingers 864, 866 are configured for sealing extension through upper and lower finger passageways 848b, 848c, respectively, of seal member 840, and through proximal passageways 838 of base 822 of transition plug 820 such that locking tabs 865, 867 extend into and engage radial openings 839 (see FIGS. 8A and 8B) to thereby secure seal clip 860 and transition plug 820 to one another with seal member 840 sandwiched in sealing engagement therebetween. The clearances between locking tabs 865, 867 and radial openings 839 are minimized so as to minimize rotational play between seal clip 860 and transition plug 820.

Clip body 862 of seal clip 860 seal member 840 includes one or more locating features 876, 878, e.g., recessed and/or protruding features, defined on or within a distal face thereof and positioned offset from central jaw drive aperture 868. Locating features 876, 878 are complementary to and configured for alignment with locating features 856, 858 of seal body 842 of seal member 840 such that, upon insertion of locking fingers 864, 866 through upper and lower finger passageways 848b, 848c to approximate seal member 840 relative to clip body 862, locating features 856, 876 and locating features 858, 878 complementarily engage one another (see FIG. 15B) to rotationally lock seal clip 860 and seal member 840 relative to one another in proper orientation.

Clip body 862 of seal clip 860 further includes a plurality of passages defined therethrough, including: a central jaw drive aperture 868; a lead wire channel 870 radially spaced from central jaw drive aperture 868 on a first side of central jaw drive aperture 868; a knife rod aperture 872 radially spaced from central jaw drive aperture 868 on a second, opposite side of central jaw drive aperture 868 in horizontal registration with central aperture 868 and lead wire cut-out 870; and four articulation cable channels 874 arranged in a square configuration wherein each articulation cable channel 874 is disposed in one of the quadrants of clip body 862. Support tube 880 is aligned with knife rod aperture 872 to guide and support knife rod 853b (FIG. 12D) into and through seal assembly 800 (FIGS. 8A-9B). Upon the above-detailed approximation and alignment of seal member 840 on seal clip 860, central jaw drive apertures 848a, 868 are aligned to receive jaw drive rod 1484 (FIG. 12D) therethrough; lead wire apertures 850a, 850b are aligned with lead wire channel 870 to receive electrical lead wires 851a, 851b (FIG. 12D) therethrough; knife rod apertures 852, 872 are aligned to receive knife rod 853b (FIG. 12D) therethrough; and articulation cable apertures 854 are aligned with articulation cable channels 874 to receive articulation cables 138 (FIG. 12D) therethrough. Knife rod aperture 872 may be defined through protruding locating feature 876 for alignment with knife rod aperture 852 upon receipt of locating feature 876 within locating feature 856 as seal member 840 is approximated relative to seal clip 860. Referring also to FIGS. 11 and 12D, lumens 828a, 828b and channels 828c of transition plug 820 are configured to receive and/or guide the various components passing through seal clip 860 and seal member 840 (e.g., jaw drive rod 1484, knife rod 853b, electrical lead wires 851a, 851b, and articulation cables 138) distally into and/or through articulating segment 136 of shaft 130 of surgical instrument 110 (see FIG. 5).

Referring generally to FIGS. 8A-15B, as detailed above, seal clip 860 is configured to engage transition plug 820 with seal member 840 sandwiched therebetween to form seal assembly 800 and retain transition plug 820, seal member 840, and seal clip 860 in engagement with one another. Further, seal assembly 800 is configured for positioning within proximal segment 134 of shaft 130 of surgical instrument 110 (FIG. 5) in sealing engagement therewith, and in engagement with proximal-most articulation link 139 of the one or more articulating components 137 of articulating section 136 of shaft 130 (see FIG. 5). In addition, seal member 840 establishes seals about the various components extending therethrough, e.g., jaw drive rod 1484, knife rod 853b, electrical lead wires 851a, 851b, and articulation cables 138. Advantageously, seal assembly 800 can itself be assembled, the various components routed therethrough, and assembled to surgical instrument 110 (FIG. 5) by hand and without the need for specialized tools for insertion, alignment, or other assembly steps.

It should be understood that various aspects disclosed herein may be combined in different combinations than the combinations specifically presented hereinabove and in the accompanying drawings. In addition, while certain aspects of the present disclosure are described as being performed by a single module or unit for purposes of clarity, it should be understood that the techniques of this disclosure may be performed by a combination of units or modules associated with, for example, a surgical system.

While several aspects of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular aspects. Those skilled in the art will envision other modifications within the scope and spirit of the claims appended hereto.

What is claimed is:

1. A surgical instrument, comprising:
a housing;
a shaft extending distally from the housing, the shaft including a proximal segment, a distal segment, and an articulating portion interconnecting the proximal and distal segments;
an end effector assembly coupled to the distal segment of the shaft;
a plurality of actuation components extending from the housing into the shaft, each actuation component of the plurality of actuation components extending through the proximal segment of the shaft to the articulating portion of the shaft, the distal segment of the shaft, or the end effector assembly; and
a seal assembly disposed within the proximal segment of the shaft, the seal assembly including:
a plug;
a clip; and
a seal member configured to establish a seal about an internal annular surface of the proximal segment of the shaft, the seal member defining a plurality of apertures therethrough, each aperture configured to sealingly receive one actuation component of the plurality of actuation components therethrough,
wherein the plug and the clip are configured to engage one another and sandwich the seal member therebetween.

2. The surgical instrument according to claim 1, wherein the clip includes at least one locking finger defining a tab at a free end thereof, the at least one locking finger configured to extend through a corresponding passageway defined within the seal member and a corresponding passageway defined within the plug such that the tab is capable of engaging an opening defined within the plug to thereby engage the plug and the clip with one another.

3. The surgical instrument according to claim 2, wherein the seal member is configured to establish a seal about the at least one locking finger extending through the corresponding passageway.

4. The surgical instrument according to claim 1, wherein the seal member includes a body defining an annular rim and at least one annular ring that protrudes radially outwardly from the annular rim, the at least one annular ring configured to facilitate sealing of the seal member about the internal annular surface of the proximal segment of the shaft.

5. The surgical instrument according to claim 1, wherein the seal member defines a central aperture configured to receive a first actuation component of the plurality of actuation components and upper and lower passageways spaced above and below the central aperture in vertical registration therewith, the upper and lower passageways configured to receive portions of the clip to enable engagement of the plug and the clip with one another.

6. The surgical instrument according to claim 5, wherein the seal member is configured to establish a seal about the first actuation component extending through the central aperture.

7. The surgical instrument according to claim 6, wherein the central aperture defines a distal portion that is enlarged compared to a proximal portion thereof.

8. The surgical instrument according to claim 1, wherein the seal member defines a central aperture configured to receive a first actuation component of the plurality of actuation components, a pair of second apertures offset on a first side of the central aperture in vertical registration with one another, and a third aperture offset on a second, oppose side of the central aperture in horizontal registration with the central aperture and collectively the pair of second apertures, the third aperture configured to receive a second actuation component of the plurality of actuation components.

9. The surgical instrument according to claim 8, wherein each second aperture of the pair of second apertures is configured to receive an electrical lead wire therethrough.

10. The surgical instrument according to claim 9, wherein the seal member is configured to establish a seal about the electrical lead wires extending through the second apertures.

11. The surgical instrument according to claim 10, wherein each of the second apertures defines a varying diameter along a length thereof.

12. The surgical instrument according to claim 8, wherein the seal member is configured to establish a seal about the first actuation component extending through the central aperture and about the second actuation component extending through the third aperture.

13. The surgical instrument according to claim 1, further comprising four additional apertures extending through the seal member, each additional aperture of the four additional apertures disposed in a different quadrant of the seal member.

14. The surgical instrument according to claim 13, wherein each additional aperture is configured to sealingly receive an additional actuation component of the plurality of actuation components.

15. The surgical instrument according to claim 13, wherein a diameter of each additional aperture is tapered along a length thereof.

16. The surgical instrument according to claim 1, further comprising a support tube secured to the clip and extending proximally therefrom, wherein one of the actuation components of the plurality of actuation components extends through the support tube.

17. The surgical instrument according to claim 1, wherein the clip and the seal member include at least one pair of complementary locating features configured to facilitate proper alignment of the seal member and the clip with one another.

18. The surgical instrument according to claim 17, wherein one actuation component of the plurality of actuation components extends through one pair of complementary locating features of the at least one pair of complementary locating features.

19. The surgical instrument according to claim 1, wherein the seal member is overmolded onto the clip.

20. The surgical instrument according to claim 1, wherein the articulating portion of the shaft includes at least one articulating link, and wherein the plug is configured to engage a proximal-most articulating link of the at least one articulating link.
